(19) United States
(12) Patent Application Publication
(10) Pub. No.: US 2022/0175657 A1
(43) Pub. Date: Jun. 9, 2022

(54) METHOD FOR TREATING HAIR AND SCALP

(71) Applicant: KAO GERMANY GMBH, Darmstadt (DE)

(72) Inventors: Iris KLAWITTER, Darmstadt (DE); Normen LIPINSKI, Darmstadt (DE)

(73) Assignee: KAO GERMANY GMBH, Darmstadt (DE)

(21) Appl. No.: 17/433,706

(22) PCT Filed: Mar. 1, 2019
(86) PCT No.: PCT/EP2019/055139

§ 371 Date: Aug. 25, 2021

(51) Int. Cl. A61K 8/9789; A61K 8/04; A61K 8/19; A61K 8/73; A61K 8/891; A61K 8/34; A61Q 5/02; A61Q 5/12; A45D 19/00

(52) U.S. Cl. CPC A61K 8/9789; A61K 8/046; A61K 8/19; A61K 8/731; A61K 8/891; A61K 8/345; A45D 2200/25; A61Q 5/12; A45D 19/0041; A61K 2800/412; A61K 2800/48; A61K 2800/548; A61K 2800/28; A61Q 5/02

(57) ABSTRACT

Methods, processes, and kits-of-parts for treating hair and scalp and increasing blood circulation of the scalp are provided. The methods and processes include applying aqueous compositions onto hairs and scalps, wherein the aqueous compositions comprise dispersed scrub particles that provide further improvement of blood circulation compared to scalps treated only with cleansing and carbon dioxide comprising compositions.

The present invention relates to a method for treating scalp and hair to increase blood circulation of the scalp.

Skin or scalp care compositions are widely used with active ingredients to protect the skin and work against wrinkles, inflammation, dandruff, hair-loss and many other medical as well cosmetic problems. The topical application of creams, gels or liquid products belong to the usual daily routine for many costumers. The mechanism of action usually depends of the concentration of active ingredients at the point of action. For an effective transport of the active molecules after topical application onto the skin, cleansing or massaging for improved blood circulation is widely used to increase effectiveness.

The physico-chemical status of head hair growing on the scalp is very much related to the health of the scalp. It has been continuously discussed in the literature the relationship between the healthy scalp and healthy hair. It is generally believed that the increased blood circulation on the scalp affects positively the health and, therefore, appearance of the hair. Blood circulation brings oxygen and nutrients to the scalp and hair follicles, which is essential for the growth. It is the general thinking that blood circulation to the scalp like fertilizer for plants in a garden.

In order to improve the scalp conditions, various products such as scalp lotions, scalp gels and products comprising carbon dioxide as the known blood circulation improving agent have been provided by various cosmetic suppliers. JP2017125003A discloses an aerosol cosmetic containing particular powder of a silicone elastomer with an average diameter of 0.01-30 μm, an oil, a non-ionic surfactant, a water-soluble thickening agent and carbon dioxide gas as propellant. The benefit of the composition is, among others, the increased blood flow, a stable foam and good spreadability with an improved non-sticky skin feel. However, there is still need for further optimized products and processes for improving scalp condition by altering the blood circulation.

The inventors of the present invention have unexpectedly found out that application of an aqueous composition comprising dispersed scrub particles provides further improvement of blood circulation compared to the scalp treated only with a cleansing and carbon dioxide comprising compositions.

Thus, the first object of the present invention is a process for treating scalp and hair comprising the steps of

a) applying a first aqueous non-cleansing composition comprising dispersed particles with a size in the range of 1 to 1000 μm measured by light microscopy, and massaging it into the scalp and hair and optionally rinsing it off,
b) applying a second aqueous cleansing composition onto scalp and hair and massaging it into scalp and hair,
c) rinsing the hair off,
d) applying a third non-cleansing aqueous aerosol composition onto hair and scalp comprising carbon dioxide as a propellant, massaging it into scalp and hair, and optionally rinsing it off,
e) optionally applying a hair conditioning composition,
f) optionally rinsing the hair off and drying.

The second object of the present invention is the use of the compositions of steps a), b), and d) as defined throughout the specification and in the claims for increasing blood circulation of human skin and/or scalp.

The third object of the present invention is a kit-of-parts comprising individually packaged compositions of steps a), b), and d) as defined throughout the specification and in the claims.

The first composition applied onto scalp and hair is an aqueous non-cleansing composition comprising dispersed particles with a size in the range of 1 to 1000 μm, measured by light microscopy. The size of the particles is preferably in the range of 5 μm to 500 μm, more preferably 50 μm to 400 μm measured by light microscopy. Suitable particles are any particles which are insoluble in the aqueous medium. The suitable particles are in an embodiment of the invention produced from one or more hydrophobic compounds having a melting point above 25° C., preferably above 30° C. and more preferably above 35° C. under atmospheric conditions. The suitable particles are selected from cellulose particles, particle of jojoba esters, the ones produced from one or more hydrophobic compounds having a melting point above 25° C., preferably above 30° C. and more preferably above 35° C. under atmospheric conditions and their mixtures.

Total concentration of dispersed particles in the first composition of step a) is in the range of 0.5 to 15% by weight, preferably in the range of 1 to 12.5% by weight, more preferably 1.5 to 10% by weight and most preferably 2 to 8% by weight, calculated to the total of the composition.

The first aqueous composition of step a) preferably comprises a thickening polymer in order to stably disperse the particles and keep the composition stable throughout the life time. Any polymer having thickening effect in an aqueous composition is in principal suitable for the purpose of the present inventions. However, better dispersablity of the particles and their improved stability is observed with polymers building a thixotropic gel in the aqueous medium. Such polymers are in principal acrylate polymers and available under the trade names Carbomer or Pemulen for various suppliers. Preferred are acrylate copolymers with and without an alkyl chain. Especially preferred are alkyl acrylate co and cross polymers such as Acrylates/C10-30 Alkyl acrylate cross polymer.

The concentration of thickening agent, may be dependent on the type of the thickener, is prinicpally in the range of 0.1 to 5% by weight, more preferably in the range of 0.2 to 4% by weight, most preferably 0.25 to 2.5% by weight, calculated to the total of the composition.

The first aqueous composition comprises furthermore preferably one or more nonionic surfactants which may serve to incorporate the sparingly soluble or insoluble compounds into the composition. These surfactants are also known as solubilizers in the art. Well known suitable nonionic surfactants as solubilizers are ethoxylated triglycerides such as hydrogenated castor oil available under the tradenames Cremophore and Eumulgin with various ethoxylation levels such as 40 and 60 ethoxy units, namely PEG 40 hydrogenated castor oil and PEG 60 hydrogenated castor oil, and cypryliccapric glycerides with ethoxylation level 4 to 10 ethoxy units such as PEG-4 Caprylic/Capric Glycerides, PEG-6 CaprylicCapric Glycerides, PEG-7 Caprylic/Capric Glycerides, and PEG-8 Caprylic/Capric Glycerides. The total concentration of the one or more nonionic surfactants in the first aqueous composition of step a) is in the range of 0.1 to 2.5% by weight, calculated to the total of the composition.

The first aqueous composition comprises one or more volatile organic solvents selected from C2 to C4 alcohols such as ethanol, propanol, isopropanol, butanol and isobutanol. The most preferred is ethanol.

The total concentration of one or more alcohol is in the range of 1 to 30% by weight, preferably 2 to 25% by weight, more preferably 5 to 20% by weight, and most preferably 1 to 30% by weight, calculated to the total of the composition.

In a preferred embodiment of the process, the first aqueous composition is massaged into the scalp and hair for a period 10 to 600 seconds, preferably 1 to 10 mins, more preferably 2 to 10 mins. The composition may be left for additional 10 min on the scalp after massaging.

The second composition of step b) comprises one or more cleansing surfactant(s), preferably selected from anionic and/or zwitterioniclamphoteric and/or non-ionic cleansing surfactants. In a preferred embodiment of the present invention, the aqueous cleansing composition comprises one or more anionic surfactant(s), one or more nonionic surfactant(s) and one or more amphoteric surfactant(s).

The total concentration of one or more surfactants in the aqueous cleansing composition of step b) is in the range of 5 to 50% by weight, preferably in the range of 8 to 25% by weight, calculated to the total of the composition.

In principal any anionic surfactant is suitable within the meaning of the present invention. Nonlimiting examples are anionic surfactants of the sulfate, sulfonate, carboxylate and alkyl phosphate type, especially, of course, those customarily used in shampoo compositions, for example, the known C₁₀-C₁₈-alkyl sulfates, and in particular the respective ether sulfates, for example, C₁₂-C₁₄-alkyl ether sulfate, lauryl ether sulfate, especially with 1 to 4 ethylene oxide groups in the molecule, monoglyceride (ether) sulfates, fatty acid amide sulfates obtained by ethoxylation and subsequent sulfatation of fatty acid alkanolamides, and the alkali salts thereof, as well as the salts of long-chain mono- and dialkyl phosphates constituting mild, skin-compatible detergents.

Additional anionic surfactants useful within the scope of the invention are α-olefin sulfonates or the salts thereof, and in particular alkali salts of sulfosuccinic acid semiesters, for example, the disodium salt of monooctyl sulfosuccinate and alkali salts of long-chain monoalkyl ethoxysulfosuccinates.

Suitable surfactants of the carboxylate type are alkyl polyether carboxylic acids and the salts thereof of the formula

R₇—(C₂H₄O)ₙ—O—CH₂COOX,

wherein R₇ is a C₈-C₂₀-alkyl group, preferably a C₁₂-C₁₄-alkyl group, n is a number from 1 to 20, preferably 2 to 17, and X is H or preferably a cation of the group sodium, potassium, magnesium and ammonium, which can optionally be hydroxyalkyl-substituted, as well as alkyl amido polyether carboxylic acids of the general formula

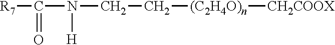

wherein R and X have the above meanings, and n is in particular a number from 1 to 10, preferably 2.5 to 5.

Such products have been known for some time and are on the market, for example, under the trade name “AKYPO®” and “AKYPO-SOFT®”.

Also useful are C₈-C₂₀-acyl isethionates, alone or in admixture with other anionic surfactants, as well as sulfofatty acids and the esters thereof.

Further suitable anionic surfactants are amino acid surfactants such as glutamate, alanin or alaninate, sarcosinate, aspartate surfactants, and mixtures thereof.

The most preferred anionic surfactants are C12-C14 alkyl ether sulphates and their salts and their mixtures.

Nonionic surfactants suitable in the cleansing compositions according to the invention are alkyl polyglucosides of the general formula

R₈—O—(R₉O)ₙ—Zₓ,

wherein R₈ is an alkyl group with 8 to 18 carbon atoms, R₉ is an ethylene or propylene group, Z is a saccharide group with 5 to 6 carbon atoms, n is a number from 0 to 10 and x is a number between 1 and 5. Suitable non-limiting and preferred examples are decyl polyglucoside, lauryl polyglucoside and cocoyl polyglucoside. It should be noted that these compounds are also known with their synonym names decyl glucoside, lauryl glucoside and cocoyl glucoside. Among these cococ glucoside is particularly preferred.

Further nonionic surfactants are, suitable for the cleansing compositions of the present invention, long-chain fatty acid dialkanolamides, such as coco fatty acid diethanolamide and myristic fatty acid diethanolamide.

Further suitable nonionic surfactants are amineoxides. Such amineoxides are state of the art, for example C₁₂-C₁₈-alkyl dimethyl amineoxides such as lauryl dimethyl amineoxide, C₁₂-C₁₈-alkyl amidopropyl or -ethyl amineoxides, C₁₂-C₁₈-alkyl di(hydroxyethyl) or (hydroxypropyl) amineoxides, or also amineoxides with ethyleneoxide and/or propyleneoxide groups in the alkyl chain. Such amineoxides are on the market, for example, under the trade names “Ammonyx®”, “Aromox®” or “Genaminox®”.

Further nonionic surfactants useful in the compositions according to invention are C₁₀-C₂₂-fatty alcohol ethoxylate. Especially suited are C₁₀-C₂₂-fatty alcohol ethers, the alkyl polyglycol ethers known by the generic terms “Laureth”, “Myristeth”, “Oleth”, “Ceteth”, “Deceth”, “Steareth” and “Ceteareth” according to the CTFA nomenclature, including addition of the number of ethylene oxide molecules, e.g., “Laureth-16”.

The average degree of ethoxylation thereby ranges between about 2.5 and about 25, preferably about 10 and about 20.

Further suitable nonionic surfactants are sorbitan surfactant. Preferably sorbitan surfactant is a sorbitan sesqui ester and more preferably it is sorbitan sesquicaprylate.

The most preferred non-ionic surfactants are alkyl polyglucosides and among those cocly glucoside or cocoyl polyglucoside is the most preferred.

Suitable amphoteric surfactants are in particular the various known betaines such as alkyl betaines, fatty acid amidoalkyl betaines and sulfobetaines, for example, lauryl hydroxysulfobetaine; long-chain alkyl amino acids, such as cocoaminoacetate, cocoaminopropionate and sodium cocoamphopropionate and -acetate have also proven suitable.

In detail, suitable betaine surfactants are of general structure

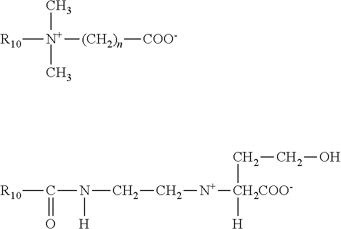

wherein R₁₀ is a C₈-C₁₈-alkyl group and n is 1 to 3;
sulfobetaines of the structure

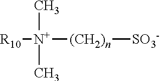

wherein R₁₀ and n are same as above;
and amidoalkyl betaines of the structure

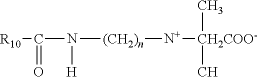

wherein R₁₀ and n are same as above.

The most preferred amphoteric surfactants are alkyl betaines such as lauryl betaine and alkyl amido betaines such as cocamidopropyl betaine.

In a further preferred form of the present invention, cleansing composition comprises at least one anionic surfactant especially of C12-C14 alkyl ether sulphate type, at least one amphoteric surfactant especially alkyl amido alkyl betaine type and at least one non-ionic surfactant especially an alkyl polyglucoside type.

In addition to the surfactants, the aqueous cleansing composition comprises one or more conditioning agents. Conditioning agents can be selected from oily substances, non-ionic substances, cationic polymers and their mixtures.

Oily substances are selected from such as silicone oils, either volatile or non-volatile, natural and synthetic oils. Among silicone oils those can be added to the compositions include dimethicone, dimethiconol, polydimethylsiloxane, DC fluid ranges from Dow Corning, arylated silicones such as phenyl trimethicone or any other silicone with up to 5 aryl, preferably phenyl, group in its molecule such as trimethyl pentaphenyl trisiloxane, natural oils such as olive oil, almond oil, avocado oil, wheatgerm oil, ricinus oil and the synthetic oils, such as mineral oil, isopropyl myristate, palmitate, stearate and isostearate, oleyl oleate, isocetyl stearate, hexyl laurate, dibutyl adipate, dioctyl adipate, myristyl myristate and oleyl erucate.

Non-ionic conditioning agents can be polyols such as glycerin, glycol and derivatives, polyethyleneglycoles known with trade names Carbowax PEG from Union Carbide and Polyox WSR range from Amerchol, polyglycerin, polyethyleneglycol mono or di fatty acid esters having general formula

R₁₁CO(OCH₂CH₂)ₙOH or

R₁₁CO(OCH₂CH₂)ₙOOCR₁₂

where R₁₁ and R₁₂ are independent from each other saturated, unsaturated or branched or non-branched alkyl chain with 7 to 21 C atoms and n is typically 2-100.

In one of the preferred form of the present invention, cleansing compositions comprise at least one additional cationic polymer as a conditioning agent. Suitable cationic polymers are those of best known with their CTFA category name Polyquaternium. Typical examples of those are Polyquaternium 1, Polyquaternium 2, Polyquaternium 4, Polyquaternium 5, Polyquaternium 6, Polyquaternium 7, Polyquaternium 8, Polyquaternium 9, Polyquaternium 10, Polyquaternium 11, Polyquaternium 12, Polyquaternium 13, Polyquaternium 14, Polyquaternium 15, Polyquaternium 16, Polyquaternium 17, Polyquaternium 18, Polyquaternium 19, Polyquaternium 20, Polyquaternium 22, Polyquaternium 24, Polyquaternium 27, Polyquaternium 28, Polyquaternium 29, Polyquaternium 30, Polyquaternium 31, Polyquaternium 32, Polyquaternium 33, Polyquaternium 34, Polyquaternium 35 and Polyquaternium 36, Polyquaternium-37, Polyquaternium 39, Polyquaternium 42, Polyquaternium 43, Polyquaternium 44, Polyquaternium 45, Polyquaternium 46, Polyquaternium 47, Polyquaternium 48, Polyquaternium-49, Polyquaternium 50, Polyquaternium 51, Polyquaternium 52, Polyquaternium 53, Polyquaternium 54, Polyquaternium 55, Polyquaternium 56, Polyquaternium 57, Polyquaternium 58, Polyquaternium 59, Polyquaternium 60, Polyquaternium 61, Polyquaternium 62, Polyquaternium 63, Polyquaternium 64, Polyquaternium 65, Polyquaternium 66, Polyquaternium 67, Polyquaternium 68, Polyquaternium 69, Polyquaternium-70, Polyquaternium 71, Polyquaternium 72, Polyquaternium 73, Polyquaternium 74, Polyquaternium 75, Polyquaternium 76, Polyquaternium 77, Polyquaternium 78, Polyquaternium-79, Polyquaternium 80, Polyquaternium 81, Polyquaternium 82, Polyquaternium 83, Polyquaternium 84, Polyquaternium 85, Polyquaternium 86 and Polyquaternium 87.

It has further been found out that especially those of cationic cellulose type polymers known as Polymer JR type from Amerchol such as Polyquaternium 10 or cationic galactomannans such as cationic guar gum known with trade name Jaguar from Rhône-Poulenc which are chemically for example Guar hydroxypropyl trimonium chloride and cationic tara gum and its derivatives known with INCI name Caesalpinia spinosa hydroxypropyltrimonium chloride, are preferred ones. Furthermore, chitosan and chitin can also be included in the compositions as cationic natural polymers. In this context reference is also made to the cationic polymers disclosed in DE 25 21 960, 28 11 010, 30 44 738 and 32 17 059, as well as to the products described in EP-A 337 354 on pages 3 to 7. It is also possible to use mixtures of various cationic polymers.

The most preferred cationic polymers are those of cationic cellulose derivatives, cationic guar gum derivatives, cationic Caesalpinia spinosa gum derivatives, polyquaternium 6, polyquaternium 7, polyquaternium 67 and polyquaternium 70.

The cationic polymers also include the quaternized products of graft polymers from organopolysiloxanes and polyethyl oxazolines described in EP-A 524 612 and EP-A 640 643.

The total concentration of one or more conditioning agents is in the range of 0.01 to 5% by weight, preferably 0.01 to 3.5% by weight, more preferably 0.05 to 2.5% and most preferably 0.1 to 1.5% by weight calculated to the total composition. Most preferred conditioning agents are cationic polymers.

The aqueous cleansing composition may comprise thickening agents in order to adjust to consistency. The well-known thickening agent especially for compositions comprising predominantly ether sulphate type of anionic surfactant is salt such as sodium and potassium chloride. Ethoxylated glycerides such as PEG-18 Glyceryl Oleate/Cocoate are also suitable thickeners.

According to the process of the present invention, in an embodiment, the cleansing composition is applied onto skin and hair and foamed by massaging in for a period of 10 to 300 seconds and rinsed off from hair.

The third non-cleansing aqueous aerosol composition is applied after the first and second aqueous compositions are rinsed off from hair and scalp. The third composition is confectioned as an aerosol product and comprises carbon dioxide as a propellant. The concentration of carbon dioxide is in the range of 0.5 to 10% by weight, preferably 1 to 5% by weight, calculated to the total of the composition.

In a further preferred embodiment of the preset invention, carbon dioxide is comprised as the sole propellant.

The non-cleansing aqueous aerosol composition of step d) comprises one or more silicone compounds, preferably having a viscosity in the range of 1 mPas to 1,000 mPas, more preferably in the range of 5 mPas to 500 mPas, most preferably in the range of 10 mPas to 300 mPas measured with a Brookfield viscometer at 60 rpm with rotor number 1 at 25° C. under atmospheric conditions. Suitable and preferred ones are dimethicones with various viscosities available from Dow Chemicals. The total concentration of the silicone compounds is in the range of 0.1 to 15%, preferably 0.25 to 10%, more preferably 0.5 to 7.5% and most preferably 1 to 7.5% by weight calculated to the total of the composition.

The non-cleansing aqueous aerosol composition of step d) comprises one or more polyol. Suitable ones are glycerol, polyethylenglcol, propylene glycol, butylene glycol, panthenol, dipropylene glycol. The total concentration of one or more polyols is in the range of 1% to 15% by weight, more preferably in the range of 1.5% to 10% by weight, calculated to the total of the composition The non-cleansing aqueous aerosol composition of step d) comprises one or more thickening polymer. In principal, any polymer having thickening effect in an aqueous composition is suitable for the purpose of the present inventions. However, better results are observed with associative polymers comprising an alkyl chain. Such polymers are in principal acrylate/methacrylate type of polymers and available from various suppliers under the trade names such as Carbomer, Pemulen and Softcare. Preferred are acrylate/methacrylate polymers with an alkyl chain. Especially preferred are alkyl acrylate co and cross polymers such as Acrylates/C10-30 Alkyl acrylate cross polymer, and lauryl acrylate/sodium methacrylate cross polymer available under the trade name Softcare ST-G from Kao Corporation.

The concentration of thickening agent in the composition of the step d), depending upon the thickener, is in the range of 0.1 to 5% by weight, more preferably in the range of 0.2 to 4% by weight, most preferably 0.25 to 2.5% by weight, calculated to the total of the composition.

The composition of step d) comprises furthermore preferably one or more nonionic surfactants which may serve to incorporate the sparingly soluble or insoluble compounds into the composition. These surfactants are also known as solubilizers in the art. Well known suitable nonionic surfactants as solubilizers are ethoxylated triglycerides such as hydrogenated castor oil available under the tradenames Cremophore and Eumulgin with various ethoxylation levels such as 40 and 60 ethoxy units, namely PEG 40 hydrogenated castor oil and PEG 60 hydrogenated castor oil, and cypryliccapric glycerides with ethoxylation level 4 to 10 ethoxy units such as PEG-4 Caprylic/Capric Glycerides, PEG-6 CaprylicCapric Glycerides, PEG-7 Caprylic/Capric Glycerides, and PEG-8 Caprylic/Capric Glycerides. The total concentration of the one or more nonionic surfactants in the first aqueous composition of step d) is in the range of 0.1 to 2.5%, preferably 0.15 to 1.25% by weight, calculated to the total of the composition.

In the process of the present invention the composition d) is applied onto scalp and hair and massaged for a period of 10 s to 600 s, preferably 30 s to 500 s and more preferably 60 s to 500 s and rinsed off from scalp and hair.

The pH of the compositions of steps a), b) and d) is in the range of 2 to 8, preferably 3 to 7, more preferably 4 to 6.5 and most preferably 4.5 to 6.5.

Any of the compositions in steps a), b) and d) may comprise one or more of the compounds selected from carboxylic acids for adjusting the pH, chelating agents, natural plant extracts, UV filters, Ubiquinones, fragrance, and preservatives.

After optionally rinsing off the composition d) from scalp and hair, optionally a hair conditioning composition is applied to the hair. Suitable compositions may be emulsions, lotions, solutions which comprises one or more conditioning agents. Any known conditioning agents as disclosed above for cleansing compositions of step b) is also suitable for the hair conditioning compositions applied after the step d). Additionally and traditionally such kind of compositions may comprise cationic, preferably quaternary ammonium, compounds. Suitable compounds are according to the general structure

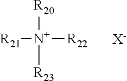

where R₂₀ and R₂₁ are a saturated or unsaturated, branched or straight alkyl chain with 8-24 C atoms, and R₂₂ and R₂₃ are independent from each other lower alkyl chain with 1 to 4 carbon atoms which may be substituted with one or two hydroxyl group, and X is chloride, bromide or methosulfate.

Non-limiting suitable long-chain quaternary ammonium compounds which are in particular cetyl trimethyl ammonium chloride, dimethyl dicetyl ammonium chloride, trimethyl cetyl ammonium bromide, stearyl trimethyl ammonium chloride, dimethyl stearyl hydroxyethyl ammonium chloride and lauryl trimethyl ammonium chloride.

The cationic compounds are comprised in the conditioning composition at a concentration in the range of 0.1 to 5% by weight, calculated to the total of the composition.

The following examples are to illustrate the invention but not to limit.

### EXAMPLE 1

First Composition (A)

| | % by weight |
|---|---|
| Cellulose scrub | 3.0 |
| Jojoba esters scrub | 3.0 |
| PEG-6 Caprylic/Capric triglycerides | 3.0 |
| Acrylates/C10-30 Alkyl acrylate crosspolymer | 1.0 |
| Ethanol | 15.0 |
| Sodium hydroxide | q.s. to pH 6.0 |
| Water | q.s. to 10 |

Second Composition (B)

| | % by weight |
|---|---|
| Sodium laureth sulphate | 10.0 |
| Cocamidopropyl betaine | 5.0 |
| Coco glucoside | 5.0 |
| PEG-18 Glyceryl cocoate/oleate | 1.0 |
| Polyquaternium-10 | 1.0 |
| Sodium hydroxide/Citric acid | q.s. to pH 5.5 |
| Water | to 100 |

Third Composition (C)

| | % by weight |
|---|---|
| Dipropylene Glycol | 4.0 |
| Dimethicone 50 CSt | 4.0 |
| Carbon Dioxide | 3.0 |
| Propanediol | 2.0 |
| PEG-60 Hydrogenated Castor Oil | 1.0 |
| Acrylates/C10-30 Alkyl Acrylate | 0.5 |
| crosspolymer | |
| Sodium hydroxide | q.s. to pH 5.5 |
| Water | q.s. to 100 |

With the above 3 compositions human skin was treated as follows: The composition A was applied in the first step at a concentration 1 g/cm² and massaged onto skin for 30 seconds. Afterwards the area was applied the Composition B, the cleansing composition, and massaged for 30 sec and rinsed off with water. Finally, the Composition C was applied at a concentration 1 g/cm² and massaged for 2 min and rinsed off with water.

Before application of the first composition A, after rinsing off the cleansing composition B and after rinsing off the Composition C photos were taken and the red color as dimension for stimulated blood circulation was evaluated.

Durability of the enhanced blood circulation was measured. Therefore, 15 panelists were treated by the above mentioned method and time was determined until the skin color returns to its usual appearance.

Analysis: Photos were analyzed with ImageJ after Optical Density Calibration. For comparison the color channels were split and the respective green channel was used for analysis of the increased red color (=increased blood circulation) of skin. The modal grey values (most frequently occurring gray value within the selection) of skin areas, where the products were applied, were measured and the difference before and after the topical application were calculated.

For the panelist the average time of the red appearance of skin was calculated for the respective products.

Following results were obtained:

| | Red colour difference |
|---|---|
| Treatment | before and after |
| Composition B | 0.016 |
| Compositions A and B | 0.029 |
| Compositions B and C | 0.045 |
| Compsitions A, B and C | 0.051 |

| Treatment | Duration of Red colour in s |
|---|---|
| Composition B | 2 |
| Compositions A and B | 7 |
| Compositions B and C | 19 |
| Compositions A, B and C | 28 |

From the above results it is beyond any doubt that the process according to the present invention shows unexpectedly the highest enhancement of the blood circulation of human skin as measured by redness evaluation.

1. A process for treating human scalp and hair, the process comprising:
a) applying a first aqueous non-cleansing composition onto a scalp and hair, massaging the first aqueous non-cleansing composition into the scalp and the hair, and optionally rinsing the first aqueous non-cleansing composition off the scalp and the hair, the first aqueous non-cleansing composition comprising dispersed particles with an average particle size in the range of 1 to 1000 μm, measured by light microscope;
b) applying a second aqueous cleansing composition onto the scalp and the hair and massaging the second aqueous cleansing composition into the scalp and the hair;
c) rinsing the hair off; and
d) applying a third non-cleansing aqueous aerosol composition onto the scalp and the hair, massaging the third non-cleansing aqueous aerosol composition into the scalp and the hair, and optionally rinsing the third non-cleansing aqueous aerosol composition off the scalp and the hair, the third non-cleansing aqueous aerosol composition comprising carbon dioxide as a propellant.

2. The process of claim 1, wherein the average particle size of the dispersed particles is in the range of 5 μm to 500 μm, measured by light microscopy.

3. The process of claim 1, wherein the first aqueous non-cleansing composition further comprises one or more dispersed particles selected from cellulose particles, particles of jojoba esters, particles being produced of one or more hydrophobic compounds having a melting point above 25° C. and at least one mixture thereof.

4. The process of claim 1, wherein a total concentration of dispersed particles in the first aqueous non-cleansing composition is in the range of 0.5 to 15% by weight, calculated to a total weight of the first aqueous non-cleansing composition.

5. The process according of claim 1, wherein the first aqueous non-cleansing composition comprises one or more thickening polymer(s), at a concentration in the range of 0.1 to 5% by weight, calculated to a total weight of the first aqueous non-cleansing composition.

6. The process of claim 1, wherein the second aqueous cleansing composition comprises one or more cleansing surfactants selected from anionic surfactants, zwitterionic/amphoteric surfactants, and non-ionic surfactants.

7. The process of claim 1, wherein a total concentration of one or more cleansing surfactants is in the range of 5 to 50% by weight, calculated to a total weight of the second aqueous cleansing composition.

8. The process of claim 1, wherein the third non-cleansing aqueous aerosol composition comprises carbon dioxide at a concentration in the range of 0.5 to 10% by weight, calculated to a total weight of the third non-cleansing aqueous aerosol composition.

9. The process of claim 1, wherein the third non-cleansing aqueous aerosol composition comprises one or more silicone compounds, having a viscosity in the range of 1 mPas to 1,000 mPas, measured with a Brookfield viscometer at 60 rpm with rotor number 1 at 25° C. under atmospheric conditions.

10. The process of claim 9, wherein the third non-cleansing aqueous aerosol composition further comprises one or more polyol, at a concentration in the range of 1% to 15% by weight, calculated to a total weight of the third non-cleansing aqueous aerosol composition.

11. The process of claim 10, wherein the third non-cleansing aqueous aerosol further comprises one or more thickening polymer selected from associative thickening polymers.

12. The process of claim 1, wherein the pH of at least one of the first non-cleansing aqueous composition, the second aqueous cleansing composition, and the third non-cleansing aqueous aerosol composition is in the range of 3 to 8.

13. The process of claim 1, wherein the third non-cleansing aqueous aerosol composition comprises one or more non-ionic surfactants, having a total concentration of surfactants in the range of 0.1% to 2.5% by weight, calculated to a total weight of the third non-cleansing aqueous aerosol composition.

14. (canceled)

15. A kit-of-parts comprising:
individually packaged compositions comprising the first non-cleansing aqueous composition, the second aqueous cleansing composition, and the third non-cleansing aqueous aerosol composition according to claim 1.

16. The process of claim 1, further comprising at least one of:
e) applying a hair conditioning composition; and
f) rinsing the hair off and drying.

17. The process of claim 2, wherein the average particle size of the dispersed particles is in the range of 50 μm to 400 μm, measured by light microscopy.

18. The process of claim 4, wherein the total concentration of dispersed particles in the first aqueous non-cleansing is in the range of 2 to 8% by weight, calculated to the total weight of the first aqueous non-cleansing composition.

19. The process of claim 1, wherein a total concentration of carbon dioxide in the third non-cleansing aqueous aerosol composition is in the range of 0.5 to 10% by weight, calculated to a total weigh of the third non-cleansing aqueous aerosol composition.

20. The process of claim 19, wherein the total concentration of carbon dioxide in the third non-cleansing aqueous aerosol composition is in the range of 1 to 5% by weight, calculated to the total of the third non-cleansing aqueous aerosol composition.

21. The process of claim 19, wherein the third non-cleansing aqueous aerosol composition is confectioned as an aerosol product comprising carbon dioxide as the sole propellant of the aerosol product.
